(19) United States
(12) Patent Application Publication
(10) Pub. No.: US 2021/0254141 A1
(43) Pub. Date: Aug. 19, 2021

(54) METHOD OF AND APPARATUS FOR ANALYZING TUMOR SUBCLONES

(71) Applicant: SEOUL NATIONAL UNIVERSITY R&DB FOUNDATION, Seoul (KR)

(72) Inventors: Sun KIM, Seoul (KR); Do Hoon LEE, Seoul (KR)

(21) Appl. No.: 16/905,083

(22) Filed: Jun. 18, 2020

(30) Foreign Application Priority Data
Feb. 18, 2020 (KR) 10-2020-0019987

(51) Int. Cl. C12Q 1/6827; C12Q 1/6806; G16B 5/00; G16B 50/30

(52) U.S. Cl. CPC C12Q 1/6827; C12Q 1/6806; G16B 5/00; G16B 50/30; C12Q 2600/156; C12Q 2545/114; C12Q 2600/106; C12Q 2600/154; C12Q 2527/125

(57) ABSTRACT

Provided are a method of and an apparatus for analyzing tumor subclones. The method and the apparatus, according to an aspect, may provide the concept of fingerprint epiloci from DNA methylation data, and may determine a composition of tumor subclones therefrom, and the inferred composition of subclones may be applied to clinical treatment of cancer patients.

### CROSS-REFERENCE TO RELATED APPLICATION

This application claims the benefit of Korean Patent Application No. 10-2020-0019987, filed on Feb. 18, 2020, in the Korean Intellectual Property Office, the disclosure of which is incorporated herein in its entirety by reference.

### BACKGROUND

### 1. Field

The present disclosure relates a method of and an apparatus for analyzing tumor subclones.

### 2. Description of Related Art

Methylation by attachment of a methyl (CH₃₋) group to DNA bases plays the most vital roles in epigenetics. DNA is made of combinations of the four nucleotides of cytosine, guanine, thymine, and adenine, and a methyl group (CH₃₋) may be added at the site (CpG) where cytosine is followed by guanine.

In human genomes, about 3% to about 4% of total cytosines is known to be methylated cytosine. Meanwhile, it is known that the degree or pattern of methylation of CpG-dinucleotides varies depending on the species of mammal and is specific to tissues.

DNA methylation may be caused by DNA methyltransferase (DNMT), which modifies human DNA. At present, three types of DNMTs have been identified in mammalian cells, and the first-discovered DNMT1 is known to function to maintain DNA methylation when DNA is synthesized during cell division. The additionally discovered DNMT3a and DNMT3b have been analyzed and found to have the ability to catalyze new methylation.

Under this technical background, various studies have been conducted on diagnosis of diseases and identification of individuals using next-generation sequencing (NGS) (Korean Patent No. 10-1629247), but there is still a lot left to do.

### SUMMARY

Provided is a method of analyzing tumor subclones in a biological sample.

Provided is a computer-readable medium on which a program for executing the method on a computer is recorded.

Provided is an apparatus for analyzing tumor subclones in a biological sample.

Additional aspects will be set forth in part in the description which follows and, in part, will be apparent from the description, or may be learned by practice of the presented embodiments of the disclosure.

An aspect provides a method of analyzing tumor subclones, the method including collecting DNA methylation data derived from a biological sample; selecting fingerprint epiloci from the collected DNA methylation data; and determining tumor subclones from the selected fingerprint epiloci.

The biological sample refers to a sample derived from an organism. The organism may be a mammal including a human. The biological sample may be derived from a body tissue or a body fluid. The tissue may be any tissue in the body, where a tumor may be generated. The body fluid may be blood, plasma, serum, urine, mucus, saliva, tears, sputum, spinal fluid, pleural fluid, nipple aspirate, lymph fluid, respiratory tract fluid, serous fluid, urogenital fluid, breast milk, lymph secretion, semen, cerebrospinal fluid, body fluid in organs, ascites, fluid from cystic tumor, amniotic fluid, or a combination thereof.

The DNA methylation data may be collected from experimental data. The experimental data may be data of detecting methylated bases by using sodium bisulfite or sodium hydrogen sulfite, or by using an antibody against 5-methylcytosine. Further, the experimental data may be collected using a Sanger sequencing method, a microeletrophoretic sequencing method, sequencing by hybridization, a clonal amplification technique, an emulsion PCR method, a polony PCR method, etc.

According to a specific embodiment, the DNA methylation data may be collected by reduced representation bisulfite sequencing (RRBS). The RRBS may be a kind of bisulfite sequencing for detecting DNA methylation that occurs in cytosine bases on genome. The RRBS may be sequencing performed using an appropriate size of genomic fragment which is produced by treatment with a specific restriction enzyme. The RRBS may be performed with respect to a genomic region with a high content of CpG on DNA, for example, CpG island. The techniques capable of detecting methylation may be used in combination.

The experimental data may be collected by a specific apparatus or kit commercially available. The apparatus may utilize next generation sequencing (NGS). The apparatus may be, for example, a 454 sequencer available from Roche, an illumina genome analyzer available from illumina, SOLID available from Applied Biosystem, or a HliScope single molecular sequencer available from Helicos biosciences, but is not limited thereto.

The DNA methylation data may be collected from a known database (DB) 31. For example, the DNA methylation data may be stored in a database (DB) which has been known in the art, such as National Center for Biotechnology Information (NCBI), Gene Expression Omnibus (GEO), European Bioinformatics Institute databases, European Nucleotide Archive, etc. Further, the DNA methylation data may be collected from new data being updated due to the development of sequencing technology.

The “clone” refers to a population of genetically identical cells or individuals, and cells included in one clone may be derived from a single cell. The “subclone” refers to a population of cells resulting from one or more genetic mutations in the clone. The genetic mutations may be epigenetic mutations. The epigenetic mutations may be methylation that occurs in cytosine bases of DNA. For example, each subclone in a tumor may be a population of cells that share a unique methylation pattern.

Several subclones may exist in a tumor. Single subclones may be derived from a single cell, and they may share any biological characteristics. Therefore, respective subclones may exhibit similar characteristics in tumor treatment or diagnosis, etc. For example, a tumor therapeutic agent may exhibit similar effects against specific subclones. According to a specific embodiment, since a composition of subclones in a tumor may be identified by using DNA methylation which is one of epigenetic mutations, a difference in responses of individuals to a therapeutic agent may be understood, and it may be usefully applied to a personalized therapy.

The term “fingerprint methylation pattern” or “fingerprint pattern” means a methylation pattern of a specific subclone.

The term “epilocus” refers to a short genomic region of about 100 bp at which methylation of a read group is mapped. The epilocus may be a region where the most frequent pattern among various methylation patterns is a fully methylated pattern or a fully unmethylated pattern.

The term “fingerprint epilocus” refers to an epilocus having the fingerprint pattern. The collected DNA methylation data may be mapped to a reference genome. The mapping may be performed by Bismark. The fingerprint epilocus may be an epilocus where read groups having a fully-methylated pattern and a fully-unmethylated pattern of CpG-dinucleotide, among the mapped read groups, account for 80% or more of the total read groups. The fingerprint epilocus may be an epilocus where CpG-dinucleotides in each mapped read have a fully methylated pattern or a fully unmethylated pattern. Further, the fingerprint epilocus may be an epilocus where each read of the mapped read groups includes 2 CpG-dinucleotides, 3 CpG-dinucleotides, 4 CpG-dinucleotides, 6 CpG-dinucleotides, 8 CpG-dinucleotides, or 10 or more CpG-dinucleotides. The fingerprint epilocus may be an epilocus where each read of the mapped read groups includes 10,000 or less CpG-dinucleotides. The fingerprint epilocus may be an epilocus where 10, 20, 30, 50, 100, 500, 1000, 2000, 5000, or 10000 reads of the mapped read groups are mapped. The fingerprint epilocus may be an epilocus where 100,000 or less reads of the mapped read groups are mapped.

The term “CpG” or “CpG-dinucleotide” refers to a state where a cytosine (C) nucleotide is followed by a guanine (G) nucleotide and they are linked together by a phosphate (p) group in DNA.

The tumor may be any benign or malignant tumor. For example, the malignant tumor is chronic myeloid leukemia, acute myeloid leukemia, chronic lymphocytic leukemia, acute lymphocytic leukemia, lung cancer, gastric cancer, colon cancer, breast cancer, bone cancer, pancreatic cancer, skin cancer, head cancer, head and neck cancer, melanoma, uterine cancer, ovarian cancer, large intestine cancer, small intestine cancer, rectal cancer, anal cancer, fallopian tube carcinoma, endometrial cancer, cervical cancer, vaginal cancer, vulva cancer, Hodgkin's disease, esophageal cancer, lymphatic cancer, bladder cancer, gallbladder cancer, endocrine gland cancer, prostate cancer, adrenal cancer, soft tissue sarcoma, urethral cancer, penile cancer, lymphocytic lymphoma, renal cancer, ureteral cancer, renal pelvic cancer, blood cancer, brain cancer, central nervous system (CNS) tumor, spinal cord tumor, brainstem glioma, or pituitary adenoma.

Before the selecting, pretreating may be further included. The pretreating may be correcting the collected DNA methylation data using a DNA methyltransferase 1-like hidden markov model (DNMT1-like HMM). The DNMT1-like HMM may be modeling of enzymatic characteristics of DNA methyltransferase 1, which is an enzyme responsible for maintaining DNA methylation in cells, using a hidden markov model (HMM). The DNMT1-like HMM may further use an expectation-maximization algorithm (EM algorithm).

The determining may be performing an operation on a binary pattern. For example, the binary pattern is a pattern where CpG-dinucleotides of a read have a fully methylated pattern and a fully unmethylated pattern. A fraction of fingerprint pattern (FF) may be drawn from the operation on the binary pattern.

The term “FF” refers to a fraction of reads regarding fingerprint pattern calculated from each fingerprint epilocus. The fraction may be a value obtained by dividing the number of reads having fully methylated CpG-dinucleotides pattern by the total number of reads mapped to the corresponding fingerprint epilocus. Relative abundance of each subclone may be estimated from the FF.

In the determining, a beta binomial mixture model may be used. In the beta binomial mixture model, when the number of fully methylated patterns and the number of fully unmethylated patterns at each fingerprint epilocus (i) are denoted by mᵢ and uᵢ, respectively, mᵢ and mᵢ+uᵢ may be parameterized by α and β. The number of parameters α and β and value thereof may be estimated by using the beta binomial mixture model.

The beta binomial mixture model may test 1 cluster to 15 clusters to select a model with the minimum Bayesian information criterion (BIC). However, the number of clusters may vary depending on the user's settings. Further, by expanding the above-described method, it is also possible to perform temporal and spatial multidimensional analysis of a single tumor sample. For example, tumor samples obtained at two different time points may be analyzed, and accordingly, the change pattern of subclones between the time points may be inferred.

The determining may be determining the number of intratumoral subclones, relative abundance of intratumoral subclones, or a combination thereof.

Another aspect provides a computer-readable medium on which a program for executing the method on a computer is recorded.

Of the terms or elements mentioned in the description of the method, the same as already mentioned are as described above.

The method may be embodied in a program that is executable in a computer, and may be implemented in a general-purpose digital computer that operates the program using the computer-readable medium. Further, a structure of data used for the above method may be recorded in a computer-readable recording medium through various means. The computer-readable recording medium includes a storage medium such as a magnetic storage medium (e.g., a ROM, a floppy disk, a hard disk, etc.) or an optically readable medium (e.g., a CD-ROM and a DVD, etc.).

Still another aspect provides an apparatus for analyzing tumor subclones, the apparatus including a collection unit for collecting DNA methylation data from a biological sample; a selection unit for selecting fingerprint epiloci from the collected DNA methylation data; and a determination unit for determining tumor subclones from the selected fingerprint epiloci.

Of the terms or elements mentioned in the description of the method or the computer-readable medium, the same as already mentioned are as described above.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram showing a hardware configuration of a computing device that integrates and analyzes DNA methylation data;

FIG. 2 is a block diagram showing a detailed hardware configuration of a processor;

FIG. 3A is an exemplary diagram for explaining a fingerprint epilocus;

FIG. 3B is a diagram showing pre-treatment of DNA methylation data;

FIG. 3C is a diagram showing a binary pattern decomposition analysis;

FIG. 3D is a diagram illustrating optimization and selection of a beta binomial distribution model;

FIG. 4A is an exemplary schematic diagram of DNA methylation maintenance for DNMT1 (black-outlined circles: template methylation, red-outlined circles: copied methylation states, empty circles: unmethylated cytosine, and filled circles: methylated cytosine);

FIG. 4B is a diagram showing characteristics of DNMT-like HMM (in the squares, a represents an “attached” hidden state, and d represents a “detached” hidden state);

FIG. 5A is a graph showing a ratio of an estimated mixing ratio to a true mixing ratio (MR) before in silico proofreading;

FIG. 5B is a graph showing a ratio of an estimated MR to a true MR after in silico proofreading;

FIG. 6A is a graph comparing error rates estimated in a fully-methylated pattern and a fully-unmethylated pattern;

FIGS. 7A to 7C are graphs showing effects of in silico proofreading on the number of detected fingerprint epiloci;

FIG. 8A is a graph showing results of applying a method according to an exemplary embodiment to simulated mixtures of three tissue cell lines (S1 to S4 each indicate that a total of four subclones were detected);

FIG. 8B is a graph showing accuracy of the estimated mixing ratio;

FIG. 9A is a graph showing results of separate analysis of samples each collected at the time of diagnosis and relapse of AML-105 samples;

FIG. 9B is a graph showing results of joint analysis of samples each collected at the time of diagnosis and relapse of AML-105 samples;

FIG. 9C is a graph showing annotation of recurrently mutated genes in the results of joint analysis of samples each collected at the time of diagnosis and relapse of AML-105 samples;

FIG. 9D is a graph showing the proportion of imprinted epiloci for each of putative epigenetic subclones at the time of diagnosis and relapse of AML-105 samples;

FIG. 9E is a schematic diagram of inferred evolutionary history of subclones of AML-105 samples;

FIG. 10A is a graph showing results of separate analysis of samples each collected at the time of diagnosis and relapse of AML-109 samples;

FIG. 10B is a graph showing results of joint analysis of samples each collected at the time of diagnosis and relapse of AML-109 samples;

FIG. 10C is a graph showing annotation of recurrently mutated genes in the results of joint analysis of samples each collected at the time of diagnosis and relapse of AML-109 samples;

FIG. 10D is a graph showing the proportion of imprinted epiloci for each of putative epigenetic subclones at the time of diagnosis and relapse of AML-109 samples; and

FIG. 10E is a schematic diagram of inferred evolutionary history of subclones of AML-109 samples.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Terms used in the present exemplary embodiments are selected from general ones that are widely used at present, as much as possible, considering functions in the present exemplary embodiments, but the terms may be changed according to the intention of those skilled in the art, precedents, or the appearance of new technology. Further, in particular cases, some terms are randomly selected, and in this case, the meanings thereof will be explained in detail in the description of the corresponding exemplary embodiment. Accordingly, the terms used herein are not just names and should be defined based on the meanings of the terms and the entire content of the present exemplary embodiments.

In the descriptions of exemplary embodiments, when a part is referred to as being “connected” to another part, it may be directly connected thereto or may be electrically connected thereto with an intervening element therebetween. Further, when a part is referred to as “including” an element, it will be understood that other elements may be further included rather than other elements being excluded unless content to the contrary is specially described. Further, the term “ . . . unit” or “ . . . module” described herein refers to a unit that may perform at least one function or operation and may be implemented utilizing any form of hardware, software, or a combination thereof.

The term “consisting of” or “including” used herein should not be construed to include all of various components or various steps described in the specification, and it should be construed that some of the components or the steps may not be included, or additional components or steps may be further included.

The description of the following exemplary embodiments should not be construed as limiting the scope, and those that may be easily inferred by a person of ordinary skill in the art should be construed as belonging to the scope of the exemplary embodiments. Hereinafter, exemplary embodiments only for illustration will be described in detail with reference to the accompanying drawings.

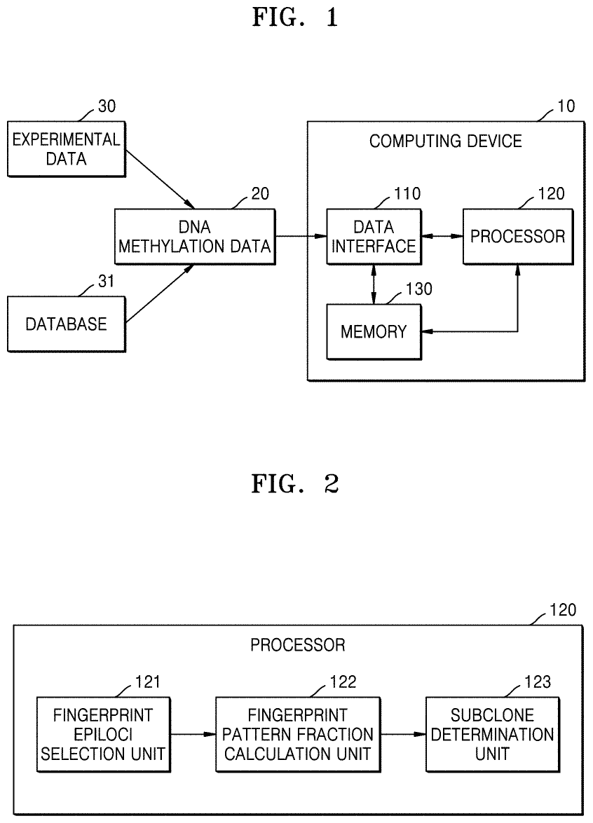

FIG. 1 is a block diagram showing a hardware configuration of a computing device 10 that integrates and analyzes DNA methylation data 20 according to a specific embodiment. In the present disclosure, the DNA methylation data 20 may be collected by using any technique capable of detecting bases where methylation occurs in DNA.

In FIG. 1, the computing device 10 may optionally include a data interface 110, a processor 120, and a memory 130. In the computing device 10 illustrated in FIG. 1, only the components related to this embodiment are shown to prevent the features of the present embodiment from being blurred, and therefore, general-purpose components other than those illustrated in FIG. 1 may be further included. The computing device 10 receives DNA methylation data 20 obtained from the experimental data 30, a database 31, or a combination thereof. The computing device 10 may finally analyze a composition of tumor subclones by analyzing the received data.

The data interface 110 may receive the DNA methylation data 20 as described above in the computing device 10. The data interface 110 may be implemented as a hardware of a wired/wireless network interface for the computing device 10 to communicate with other external devices.

The memory 130 may be a hardware for storing data to be processed in the computing device 10 and results of processing. For example, the memory 130 may include a memory chip such as a random access memory (RAM), a read only memory (ROM), etc., or a storage such as a hard disk drive (HDD), a solid state drive (SSD), etc. The memory 130 may store the DNA methylation data 20 obtained by the data interface 110. The memory 130 may store data of fingerprint epilocus selection, data of fraction of fingerprint pattern, data of subclones, etc., analyzed by the processor 120.

The processor 120 may be a hardware for analyzing intratumoral subclones using the DNA methylation data 20. The processor 120 is a module implemented by one or more processing units, and may be implemented by a combination of a microprocessor having an array of multiple logic gates and a memory module in which a program executed in the microprocessor is stored. The processor 120 may be implemented in the form of a module of an application program.

Tumor subclone information analyzed by the processor 120 may be transmitted to an external device such as a display device or another computing device, or an external network such as internet or public databases, through the data interface 110.

FIG. 2 is a block diagram showing a detailed hardware configuration of the processor of FIG. 1. Referring to FIG. 2, the processor 120 may optionally include a fingerprint epilocus selection unit 121, a fingerprint pattern fraction calculation unit 122, and a subclone determination unit 123. In the processor 120 illustrated in FIG. 2, only the components related to this embodiment are shown to prevent the features of the present embodiment from being blurred, and therefore, general-purpose components other than those illustrated in FIG. 2 may be further included. The fingerprint epilocus selection unit 121, the fingerprint pattern fraction calculation unit 122, and the subclone determination unit 123 are only divided into separate independent names according to respective functions, and they may be implemented as one processor 120. Alternatively, each of the fingerprint epilocus selection unit 121, the fingerprint pattern fraction calculation unit 122, and the subclone determination unit 123 may correspond to one or more processing modules in the processor 120. Alternatively, the fingerprint epilocus selection unit 121, the fingerprint pattern fraction calculation unit 122, and the subclone determination unit 123 may correspond to separate software algorithm units separated according to respective functions. In other words, the implementation form of the fingerprint epilocus selection unit 121, the fingerprint pattern fraction calculation unit 122, and the subclone determination unit 123 in the processor 120 is not limited to any one of them.

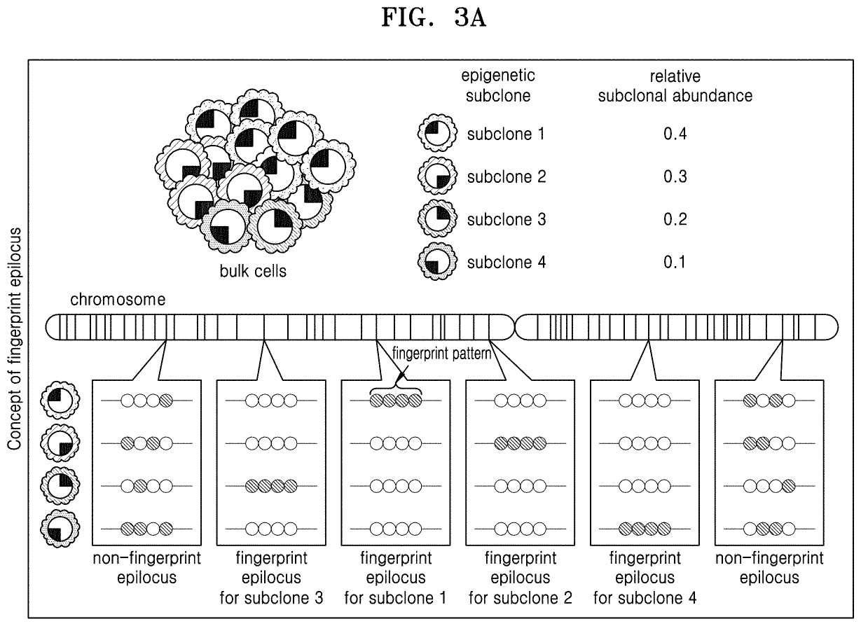

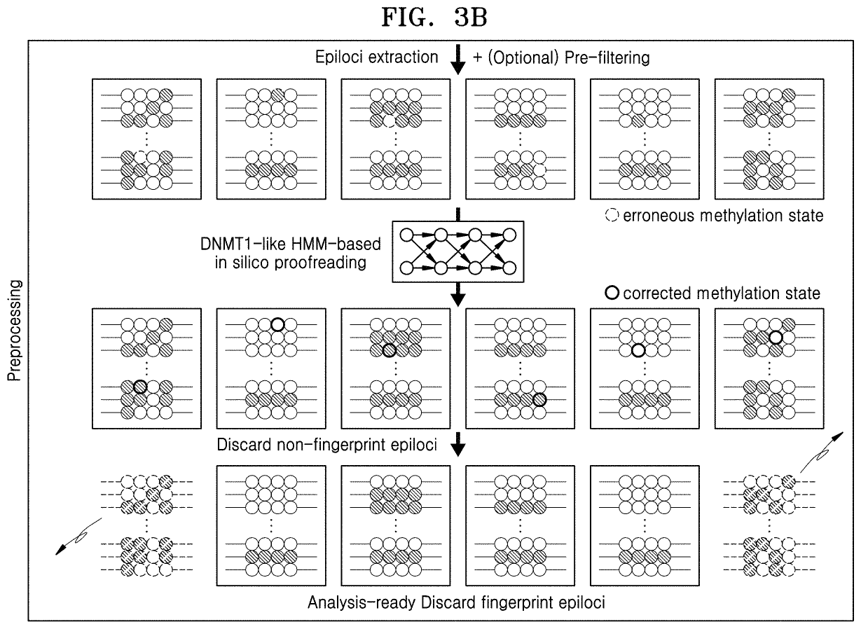

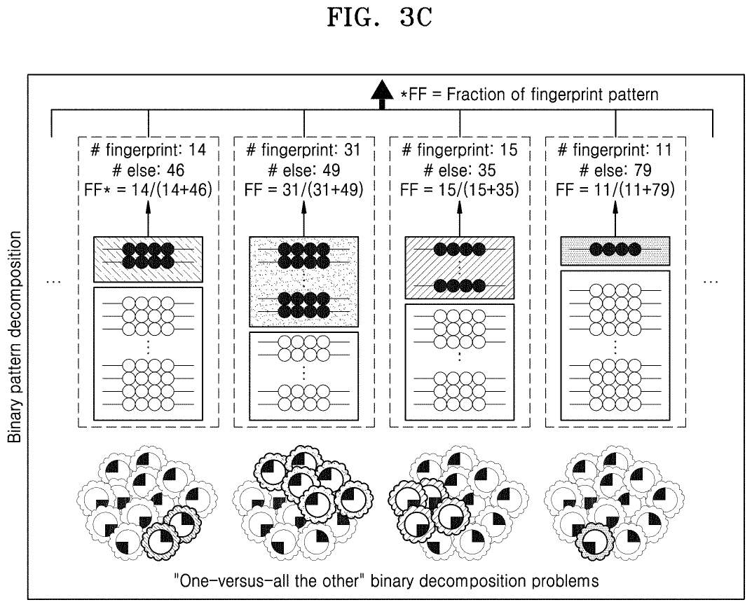

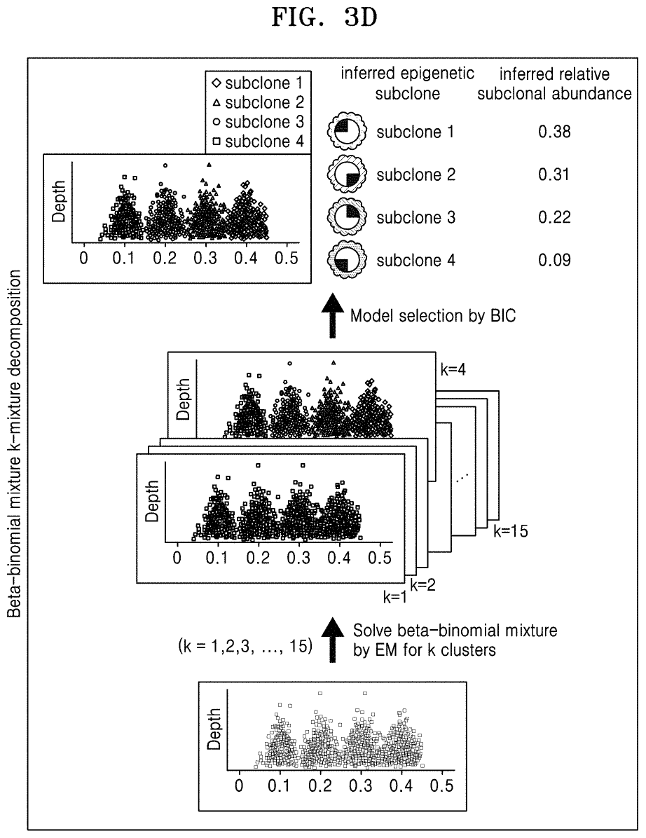

FIGS. 3A to 3D shows overall flow charts of a method of analyzing tumor subclones from DNA methylation data according to one embodiment.

FIG. 3A is an exemplary diagram for explaining a fingerprint epilocus. The tumor sample of FIG. 3A may be collected from blood, and may be collected from a tumor tissue of a body in an invasive manner. White and black circles represent unmethylated and methylated regions, respectively. The region may be a CpG-rich region. FIG. 3A illustrates four types of subclones in a tumor, and relative abundance of each subclone. In addition, fingerprint epiloci of subclones 1, 2, 3 and 4 are shown at the bottom of FIG. 3A. For simplicity of the method, only fully methylated fingerprint patterns are shown as fingerprint epiloci in FIG. 3A. However, fully unmethylated regions may also be considered as fingerprint epiloci, and any of using DNA methylation to analyze the composition of subclones may be used as fingerprint epilocus.

FIG. 3B shows fingerprint epilocus selection of the DNA methylation data 20. In the selection, pretreatment of pre-filtering may be optionally performed. A specific read may be pre-filtered out if it did not meet any one or more of specific criteria. First, the two most frequent patterns together should be fully methylated or fully unmethylated. Second, reads having the two most frequent patterns should account for 50% or 80% or more of reads mapped at the epilocus.

The selection may include selecting the fingerprint epilocus. RRBS data of tumor samples may be mapped to a reference genomic sequence using Bismark. From the mapping results, fingerprint epiloci may be extracted. For example, a region where 20 or more of the mapped reads with four or more CpG-dinucleotides are mapped may be fingerprint epilocus. The numbers of CpG-dinucleotides and reads may be appropriately selected by those skilled in the art. The fingerprint epilocus may be a region where CpG of each read may be fully methylated or fully unmethylated. In the selection, obvious non-fingerprint epiloci may be discarded.

In FIG. 3B, methylation data of the selected fingerprint epiloci may include many errors. The errors may be caused by a relatively high error rate of an intracellular mechanism responsible for DNA methylation maintenance, an error in bisulfite treatment, an error in sequencing, etc. Since the number and composition of the tumor subclone are estimated by using DNA methylation, the errors may reduce the estimation.

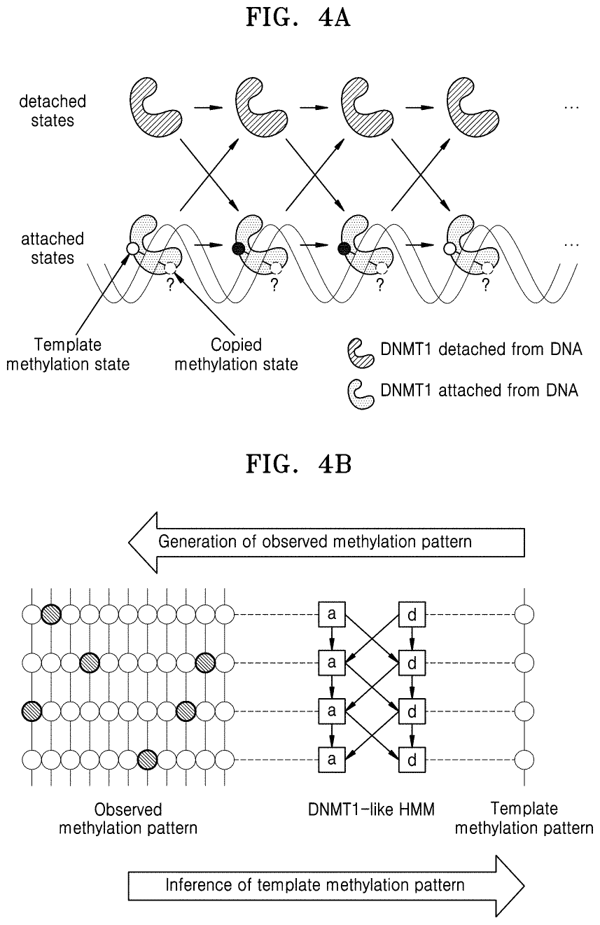

FIGS. 4A and 4B show DNA methyltransferase 1 (DNMT1)-like hidden markov model (HMM) for reducing errors of DNA methylation data in the selection. The DNMT-like HMM may infer errors by a hidden markov model using two states of an “attached (a)” state of DNMT1 to DNA and a “detached (d)” state of DNMT1 from DNA.

FIG. 4A shows an exemplary schematic diagram of DNA methylation maintenance for DNMT1. Copied methylation states may be yet unknown. DNMT1 may be in two states: detached from DNA (green) and attached to DNA (red). The probability of transition between states may be either an experimentally obtained value or an appropriate known value. Errors may be solved by a method of putting groups of observed DNA methylation patterns to HMM model, and then identifying the most likely template methylation patterns using an expectation-maximization algorithm (EM algorithm).

FIG. 4B shows characteristics of DNMT-like HMM. In FIG. 4B, methylation patterns may be observed leftwards arrow, and probability thereof may be defined, and probabilities of template methylation patterns may be inferred from observed methylation patterns.

When the number of fully methylated patterns and the number of fully unmethylated patterns at each fingerprint epilocus (i) are denoted by mᵢ and uᵢ, respectively, mᵢ and mᵢ+uᵢ may be modeled with beta-binomial distribution parameterized by α and β. By considering m and u values of all fingerprint epiloci at the same time, the solution of the beta binomial mixture model may be solved to eventually estimate the number of parameters α and β and values thereof. The mixture model may be chosen by selecting a model with the minimum Bayesian information criterion (BIC) among 1 cluster to 15 clusters tested.

Referring to FIG. 3, FIG. 3C shows a binary pattern decomposition analysis. Fingerprint epiloci which are prepared to be analyzed may be fully-methylated patterns and fully-unmethylated patterns.

FIG. 3D shows optimization and selection of a beta binomial distribution model. All the solutions of binary pattern decomposition may be merged into a single beta-binomial mixture model. Each k-cluster model may be fit by EM algorithm. k may be given an integer of 1 to 15. A model with the minimum BIC may be selected, but the number of models to be tested may vary depending on the purpose. The obtained subclonal results may be used for further analyses such as functional annotation.

Hereinafter, the present disclosure will be described in more detail with reference to exemplary embodiments. However, these exemplary embodiments are only for illustrating the present disclosure, and the scope of the present disclosure is not limited to these exemplary embodiments.

### Example 1. Examination of in Silico Proofreading Effect

Effect of in silico proofreading on accuracy of the estimated size of subclones was assessed.

In detail, raw RRBS data of a fully methylated cell line and a fully unmethylated cell line were collected. Two RRBS data were mixed to simulate a mixture of epigenetically homogeneous cells. Each of the two raw data were subsampled with 10%, 20%, 30%, 40%, 50%, 60%, 70%, 80%, 90% of reads to generate benchmark mixtures of the two cell lines. Subsequently, corresponding pairs of subsampled data were concatenated such that their mixing ratio (MR) summed up to 100%. For example, 30%-subsampled fully methylated cell line RRBS data were joined together with 70%-subsampled fully unmethylated cell line RRBS data. This entire step was repeated for 10 times. Then, accuracy of MR estimates with or without in silico proofreading was examined.

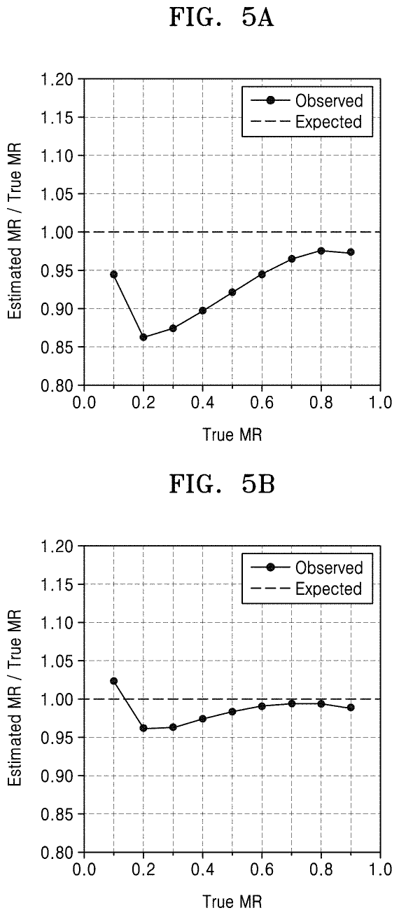

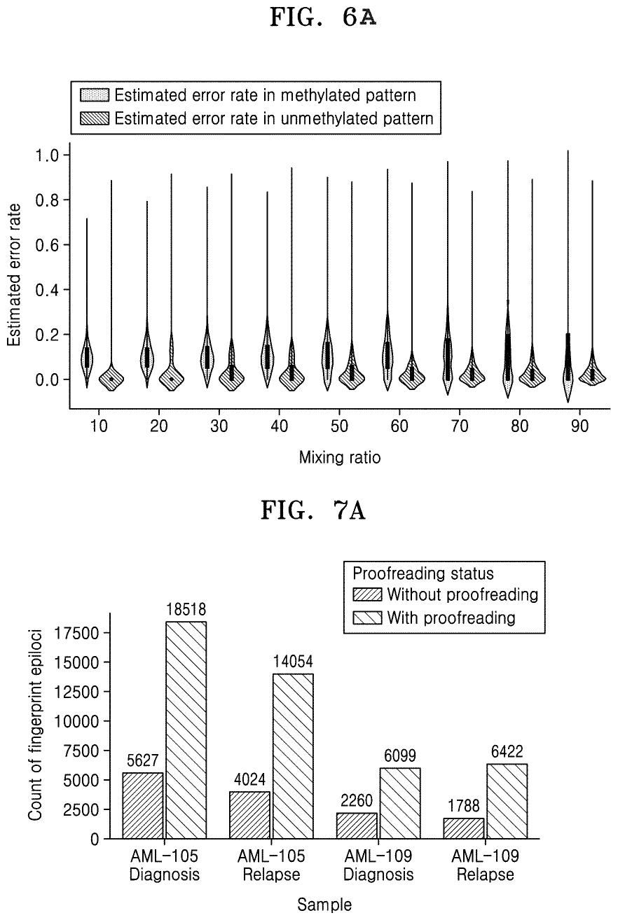

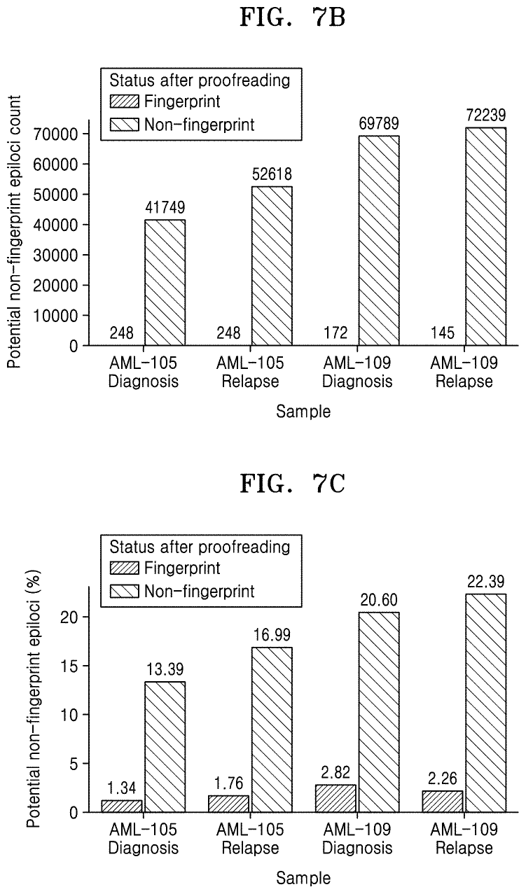

As a result, as shown in FIG. 5A, the treatment without in silico proofreading resulted in markedly biased estimations, which consistently underestimated MR. The worst estimation was for the MR of 20%, where a ratio of estimated MR to true MR was 0.86. As shown in FIG. 5B, the estimated MR after in silico proofreading were found to be more calibrated to correct estimations. The worst estimation after in silico proofreading was for the MR of 20%, and a ratio of estimated MR to true MR was 0.96. As shown in FIG. 6, in silico proofreading greatly reduced the biased error rate of methylation patterns, which was significantly higher for fully methylated patterns than for fully unmethylated patterns. Further, as shown in FIG. 7, it was found that in silico proofreading considerably increased the number of fingerprint epiloci for the inference of epigenetic subclones with marginal introduction of artificial fingerprint epiloci.

### Example 2. Examination of Practical Putative Effect on Cell Lines

Epigenomic reprogramming such as methylation may shape distinct methylation landscape for each cell type from different cell lineage. Therefore, when the method or apparatus according to a specific embodiment will be able to analyze the composition or number of tumor subclones, only when it is able to practically distinguish cell lines using DNA methylation data. To evaluate the effect of the method or apparatus according to a specific embodiment, more realistic benchmark mixtures were analyzed by mixing cell line RRBS data established from various tissues.

In detail, three cell line RRBS data were chosen from ENCODE project (Varley et al., 2013). The three cell lines were an MCF10A-Er-Src cell line derived from non-tumorigenic epithelial cells of the mammary gland, a GM06990 B-lymphocyte cell line derived from lymphoblastoid, and a T-47D cell line derived from mammary ductal carcinoma.

In this experiment, raw RRBS data of three cell lines were independently processed and mapped to the reference genome. Then, the epiloci which appeared in all of three alignment results and had 20 or more mapped reads were retained for the mixing procedure. For each epilocus, simulated sequencing depth d was sampled from NegBin(5, 0.03) with constraint d_20. P1, P2, P3 which are MRs were randomly sampled from Dirichlet(3, 3, 3), and for each epilocus, Pᵢd reads were sampled from each of the three data. The entire mixing was repeated twice to generate two independent mixtures as in Table 1 below.

TABLE 1

| | MCF10A-Er-Src | GM06990 | T-47D |
|---|---|---|---|
| Mixture 1 | 20.4% | 30.6% | 49.0% |
| Mixture 2 | 41.7% | 17.4% | 40.9% |

Each cell line was supposed as a putative subclone in the mixture, and the method according to a specific embodiment was used to estimate the number and abundance of the subclones from their mixed methylation patterns in the two mixtures.

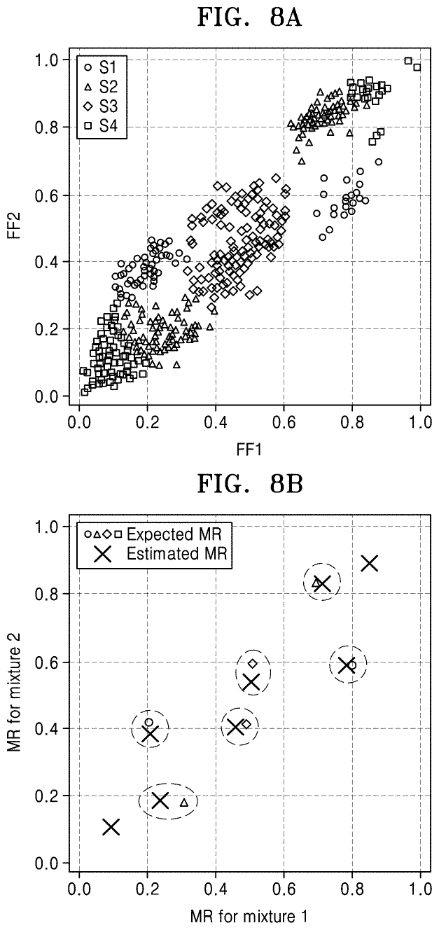

As a result, as shown in FIG. 8A, four subclones were identified. Regarding the average FF of each cluster as MR estimate, it was observed that the MR estimates of respective subclones reasonably represented the true MRs, as shown in Table 2 below and FIG. 8B. By comparing MR estimates with true MRs, it was confirmed that subclones 1, 2 and 3 represent MCF10A-Er-Src, GM06990, and T-47D, respectively. Meanwhile, subclone 4 may be generated during sequencing procedures, or it may be a new subclone originated from clonal evolution of the cell line.

TABLE 2

| | Subclone 1 | Subclone 2 | Subclone 3 | Subclone 4 |
|---|---|---|---|---|
| Mixture 1 | 20.7% | 23.6% | 45.7% | 9.2% |
| Mixture 2 | 38.0% | 18.3% | 40.0% | 10.5% |

### Example 3. Detection of Epigenetic Subclone from Acute Myeloid Leukemia Sample

To examine whether clinically meaningful observations may be drawn, the method according to a specific embodiment was assessed by applying the method to acute myeloid leukemia (AML) samples.

For each subject, a couple of samples were taken at time points of diagnosis and relapse, respectively and sequenced by RRBS. Two samples were analyzed, which resulted in 3.13 inferred subclones on average. In this experiment, analysis was performed for subjects AML-105 and AML-109, which seemed to have 5 subclones, respectively. Results of microscopic inspection revealed that each of the samples had relatively normal cytogenetic properties, except for AML-105 relapse sample, which had a small fraction of 10% or less harboring genomic deletion in q-arm of chromosome 7. Moreover, no significant CNA was detected from WES data of those samples. Therefore, it was confirmed that the CNA of the samples would not affect the analysis.

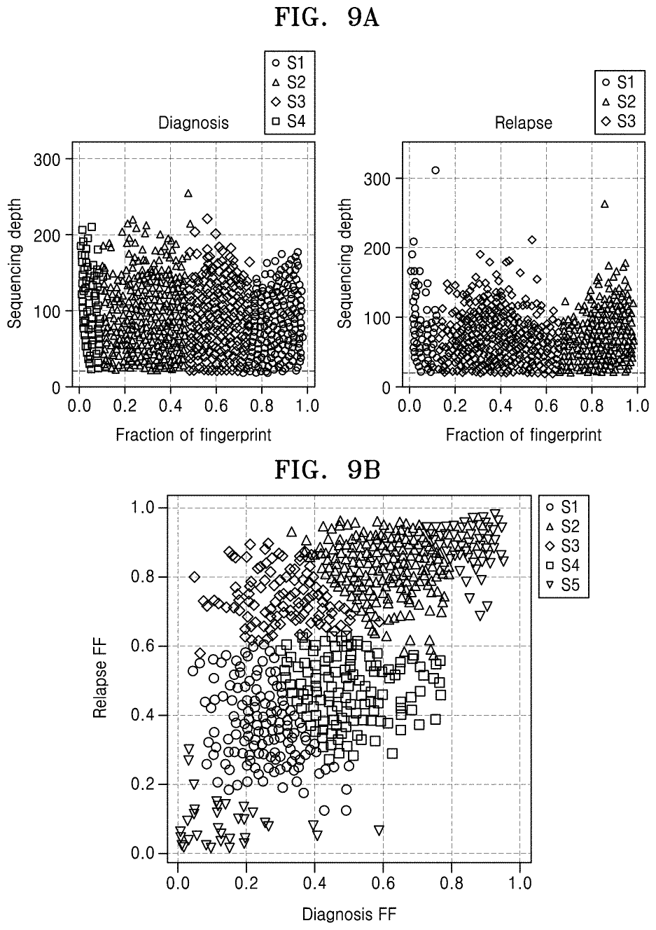

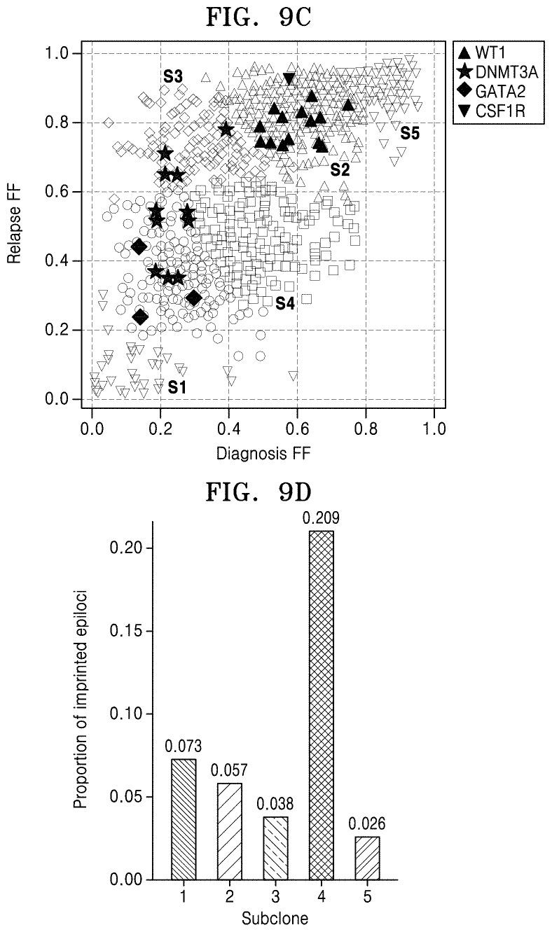

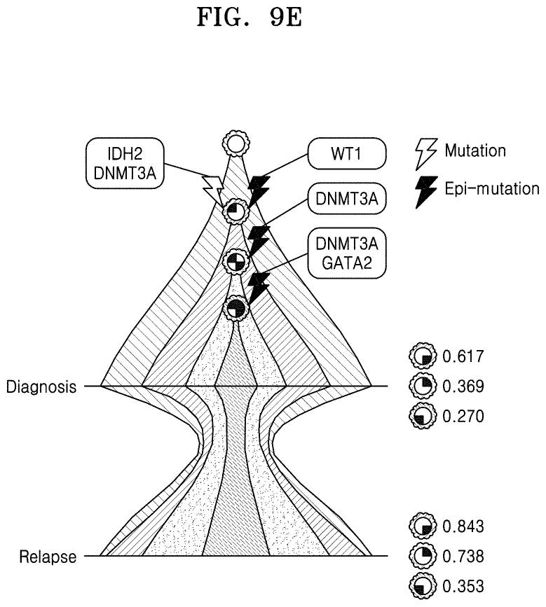

FIG. 9A shows results of initial separate analysis of diagnosis and relapse samples of AML-105. Four and three putative epigenetic subclones were found, respectively. However, the two-sample joint analysis identified five epigenetic subclones, as shown in FIG. 9B. This result emphasizes the necessity of multi-sample joint analysis to achieve a reasonable result of subclonal inference. Further, independent analysis of variant allele frequency (VAF) from WES data revealed that the subclonal abundance inferred by VAFs of heterozygous somatic mutations within isocitrate dehydrogenase 2 (IDH2) and DNA methyltransferase 3 alpha (DNMT3A) was concordant with the subclonal abundance estimates of subclone 2 (0.61-0.84) identified by the method according to a specific embodiment. FIG. 9C shows results of annotation of recurrently mutated genes in AML in the results of joint analysis of two samples. Each mark of FIG. 9C denotes epilocus which overlaps with the corresponding gene or its promoter. FIG. 9D shows the proportion of imprinted epiloci for each of putative epigenetic subclones. Notably, 20.9% of epiloci assigned to subclone 4 were annotated to known imprinted genes. Therefore, subclone 4 was excluded from further analyses. FIG. 9E is a schematic diagram of inferred evolutionary history of subclones, in which possible evolutionary history of identified epigenetic subclones is shown. Mutations and epi-mutations characterizing each subclone are represented. The horizontal black line represents the time point at which the two samples were taken. Subclone 3 (green) was found to undergo rapid clonal expansion from relative abundance 0.369 to 0.738 after chemotherapy.

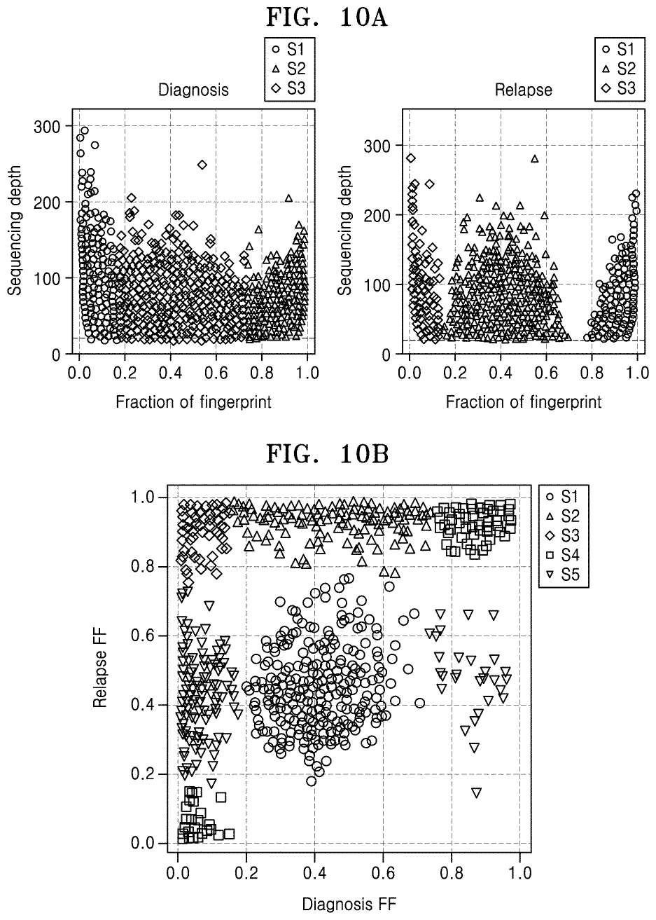

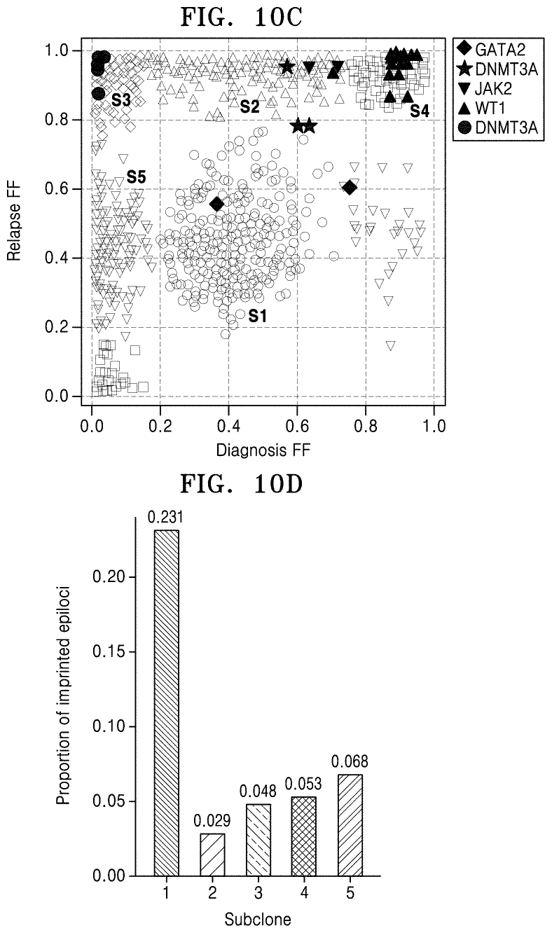

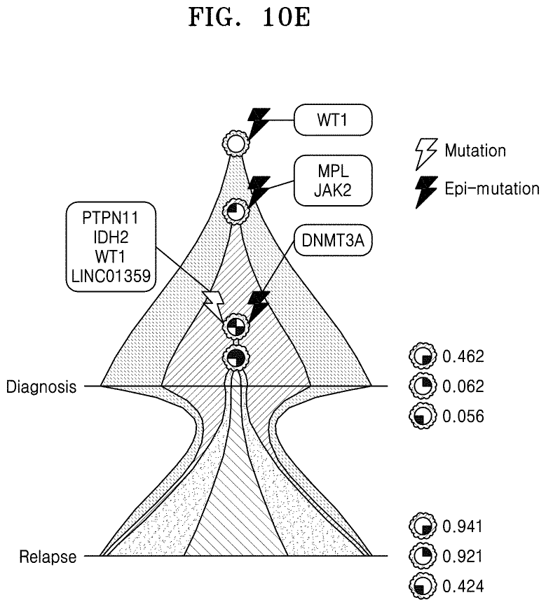

FIG. 10A shows results of separate analysis of samples each collected at the time of diagnosis and relapse of AML-109. The method according to a specific embodiment detected three candidate epigenetic subclones for each sample. FIG. 10B shows results of joint analysis of two samples, in which six epigenetic subclones were identified. FIG. 10C shows results of annotation of five recurrently mutated genes in AML in the results of joint analysis. FIG. 10D shows the proportion of imprinted epiloci for each of epigenetic subclones, in which subclone 1 was excluded from further analyses, since 23.1% of epiloci assigned to subclone 1 were imprinted. FIG. 10E is a schematic diagram of inferred evolutionary history of subclones, in which possible evolutionary history of identified epigenetic subclones is shown. Mutations and epi-mutations characterizing each subclone are represented. The horizontal black line represents the time point at which the two samples were taken. Subclone 3 (green) was found to undergo rapid clonal expansion from relative abundance 0.062 to 0.921 after chemotherapy.

The existing subclone detection technologies which are limited to genomic data are expanded and allowed to utilize epigenomic data, and ultimately, it is possible to detect subclones in various tumors by integrating genomic and epigenomic data. In addition, when the detected intratumoral subclones are applied to clinical treatment, they may contribute to predicting efficacy of chemotherapy, predicting prognosis of cancer patients, selecting appropriate anticancer drugs, etc.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of analyzing tumor subclones, the method comprising:
collecting DNA methylation data derived from a biological sample;
selecting fingerprint epiloci from the collected DNA methylation data; and
determining tumor subclones from the selected fingerprint epiloci.

2. The method of claim 1, wherein the biological sample is derived from tissue, blood, plasma, or serum of a body.

3. The method of claim 1, wherein the DNA methylation data is collected by reduced representation bisulfite sequencing (RRBS).

4. The method of claim 1, wherein the selecting is selecting fingerprint epiloci where each read of mapped read groups comprises 4 or more CpG-dinucleotides.

5. The method of claim 1, wherein the selecting is selecting fingerprint epiloci where 20 or more reads of mapped read groups are mapped.

6. The method of claim 1, wherein the selecting is selecting fingerprint epiloci where CpG-dinucleotides of mapped read groups are fully methylated or fully unmethylated.

7. The method of claim 1, further comprising pretreating the collected DNA methylation data using a DNA methyltransferase 1-like hidden markov model (DNMT1-like HMM), before the selecting.

8. The method of claim 7, wherein the DNMT1-like HMM further uses an expectation-maximization algorithm (EM algorithm).

9. The method of claim 1, wherein the determining is performing an operation on a binary pattern.

10. The method of claim 1, wherein the determining uses a beta binomial mixture model.

11. The method of claim 10, wherein the beta binomial mixture model is for selecting a model with the minimum Bayesian information criterion (BIC).

12. The method of claim 1, wherein the determining is determining the number of intratumoral subclones, relative abundance of intratumoral subclones, or a combination thereof.

13. A computer-readable medium on which a program for executing the method of claim 1 on a computer is recorded.

14. An apparatus for analyzing tumor subclones, the apparatus comprising:
a collection unit for collecting DNA methylation data from a biological sample;
a selection unit for selecting fingerprint epiloci from the collected DNA methylation data; and
a determination unit for determining tumor subclones from the selected fingerprint epiloci.

15. The apparatus of claim 14, wherein the selection unit selects fingerprint epiloci where CpG-dinucleotides of mapped read groups are fully methylated or fully unmethylated.

16. The apparatus of claim 14, further comprising a pretreatment unit for pretreating the DNA methylation data collected from the collection unit using a DNA methyltransferase 1-like hidden markov model (DNMT1-like HMM).

17. The apparatus of claim 14, wherein the determination unit uses a beta binomial mixture model.

18. The apparatus of claim 17, wherein the beta binomial mixture model is for selecting a model with the minimum Bayesian information criterion.
